(19) United States
(12) Patent Application Publication
(10) Pub. No.: US 2010/0262023 A1
(43) Pub. Date: Oct. 14, 2010

(54) POWER APPROACH TO BIOMEDICAL SIGNAL ANALYSIS

(76) Inventor: Michael L. Williams, Melrose, MA (US)

Correspondence Address: Mark J. Pandiscio;Pandiscio & Pandiscio, P.C., 470 Totten Pond Road, Waltham, MA, 02451-1914, US

(21) Appl. No.: 12/750,513

(22) Filed: Mar. 30, 2010

Related U.S. Application Data
(60) Provisional application No. 61/211,422, filed on Mar. 30, 2009.

(51) Int. Cl. A61B 5/02; A61B 5/0476; A61B 5/05

(52) U.S. Cl. 600/500; 600/544; 600/554; 600/300

(57) ABSTRACT

A consideration of electromagnetic energy transfer following from Poynting's theorem leads to a power signal that facilitates the detection of biomedical pulses. This power signal is derived from and is complementary to the measured biomedical voltage signals. The method may be applied to F-wave signals obtained from nerve conduction studies as well as other biomedical signals. Among other things, this power signal is useful in latency determination.

### REFERENCE TO PENDING PRIOR PATENT APPLICATION

This patent application claims benefit of pending prior U.S. Provisional Patent Application Ser. No. 61/211,422, filed Mar. 30, 2009 by Michael L. Williams for POWER APPROACH TO BIOMEDICAL SIGNAL ANALYSIS (Attorney's Docket No. NEURO-46 PROV), which patent application is hereby incorporated herein by reference.

### FIELD OF THE INVENTION

This invention relates to biomedical apparatus and procedures in general, and more particularly to biomedical apparatus and procedures for analyzing biomedical signals.

### BACKGROUND OF THE INVENTION

Biomedical activity is routinely studied by recording voltage measurements which are reflective of complex biochemical processes and then signal processing the acquired signals so as to assess the underlying biomedical activity. More particularly, biomedical activity is routinely studied by recording voltage measurements continuously in time and then signal processing the acquired data. Measurements are typically recorded from quiescent activity or after an external stimulus. In either case, the output often exhibits pulsed behavior corresponding to the nature of the underlying biomedical processes. By analyzing the attributes of the recorded voltage pulses, assessments can frequently be made of the underlying biomedical processes.

By way of example but not limitation, nerve conduction studies (NCS) are frequently performed to detect and evaluate focal and systemic neuropathies of peripheral nerves and spinal nerve roots. In such studies, nerves are electrically stimulated so as to evoke electrical responses. The attributes of the evoked electrical responses (e.g., the onset of pulse, the waveform of the pulse, etc.) can be used to evaluate neuropathies.

Unfortunately, there can be many spurious events which may give an erroneous indication of the onset of the pulse (i.e., a departure from a baseline) or give an erroneous indication of the waveform of the pulse, etc. These spurious events can include stimulus artifacts, contamination artifacts from a co-stimulated nerve, muscle artifacts, external noise sources, and the like. Unfortunately, an incorrect indication of of the onset of the pulse (i.e., the departure from a baseline), or an incorrect indication of the waveform of the pulse, etc. can result in an incorrect diagnosis of neurological function and/or require human intervention in a process that could otherwise be more automated.

Similar situations can occur with other biomedical signals as well.

As a result, a primary object of the present invention is to provide a new and improved method and apparatus for more accurately determining the attributes of a detected electrical signal, including determining the onset of the pulse (i.e., the point in time of a departure from a baseline voltage signal) in order to better assess event onset, waveform morphology (e.g., pulse width, pulse amplitude, etc.), etc.

### SUMMARY OF THE INVENTION

The present invention provides a new and improved method and apparatus for more accurately determining the attributes of a detected electrical signal, including determining the onset of the pulse (i.e., the point in time of a departure from a baseline voltage signal) in order to better assess event onset, waveform morphology (e.g., pulse width, pulse amplitude, etc.), etc.

The arrival of a voltage pulse is more fully described as the arrival of a pulse of electromagnetic energy. In general, a measurement involves the transfer of energy from the system being studied to the measurement device. The present invention considers biomedical voltage measurements within the context of electromagnetic energy transfer.

More particularly, the biomedical voltages which are being measured are the result of complex biochemical processes. Regardless of the origins of the voltage signal, the electromagnetic energy transfer is governed by the Poynting vector. By examining Poynting's theorem with respect to the information available from biomedical voltage measurements, reliable information can be gleaned from the biological voltage measurements. In particular, although the total power delivered is not calculable, a component of the power is derivable from the voltage measurements. The time dependence of this power component mirrors the time dependence of the overall power delivered. Thus, this power component can provide information about the time arrival of pulses resulting from biomedical activity. This information supplements the information contained in the raw voltage signal, and can permit more accurate analysis of biomedical signals.

The new method of the present invention can be applied to signal responses obtained from nerve conduction studies. The latency is the time of arrival of a nerve pulse following the stimulation of the nerve. Latency assignment is an important nerve conduction signal processing task. Using the new method of the present invention, latency assignment can be achieved in problematic cases where latency assignment fails when solely processing the original voltage signal. The new method of the present invention can also be applied to analyze waveform morphology (e.g. pulse width, pulse amplitude, etc.), etc.

The new method of the present invention can also be applied to the analysis of other biomedical signals.

In one preferred form of the present invention, there is provided a method for studying biomedical processes reflected in a recorded biomedical voltage signal, wherein the method comprises:

obtaining the recorded biomedical voltage signal;

deriving a power signal from the recorded biomedical voltage signal; and

identifying attributes in the corresponding power signal so as to provide information about the underlying biomedical processes.

In another preferred form of the present invention, there is provided a system for studying biomedical processes reflected in a recorded biomedical voltage signal, wherein the system comprises:

first apparatus for obtaining and storing a recorded biomedical voltage signal;

second apparatus for deriving a power signal from the recorded biomedical voltage signal; and

third apparatus for identifying attributes in the corresponding power signal so as to provide information about the underlying biomedical processes.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic view showing a parallel RC circuit with a driving voltage V;

FIG. 2 is a schematic view showing a series of clean F-wave voltage signals (blue) overlaid with their Stored Voltage Powers (SVPs) (red)—latencies calculated from the voltage are marked with an ‘o’, those calculated from the Stored Voltage Powers (SVPs) are marked with an ‘x’;

FIG. 3 is a schematic view showing a series of F-wave voltage signals (blue) in the presence of background activity and their Stored Voltage Powers (SVPs) (red)—calculated latencies are marked with an ‘x’;

FIG. 4 is a schematic view showing a series of F-wave voltage signals (blue) with their Stored Voltage Powers (SVPs) (red)—calculated latencies are marked with an ‘x’;

FIG. 5 is a schematic view showing an ensemble of F-wave signals for a tibial nerve conduction test with low background activity and the calculated Stored Voltage Powers (SVPs);

FIG. 6 is a schematic view showing an ensemble of F-wave signals for a tibial nerve conduction test with high background activity and the calculated Stored Voltage Powers (SVPs);

FIG. 7 is a schematic view showing an ensemble of F-wave signals for a tibial nerve conduction test with 60 Hz contamination and the calculated Stored Voltage Powers (SVPs); and

FIG. 8 is a schematic view showing the measured F-wave signals riding on a motor signal for an ulnar nerve conduction test and the calculated Stored Voltage Powers (SVPs).

### DETAILED DESCRIPTION OF THE PREFERRED EMBODIMENTS

### The Power Method In General

Electromagnetic Energy

Although voltage is a common biomedical measurement, in electromagnetic theory the fields are considered to be the more fundamental quantities. Time-varying voltages measured at a distance from the source of the electromagnetic activity are the result of propagating electric and magnetic fields. The propagating fields deliver energy from the sources to the surrounding volume. In biological systems, chemical reactions and ionic movement are generally the sources of the bioelectrical activity. Energy generated by these processes is propagated by the fields to surrounding tissue. A voltage measurement is the interception of a fraction of the propagated energy. A time-varying voltage is a consequence of time-varying energy delivery to the measurement location.

Biomedical voltage measurements are carried out to obtain information about the underlying source activity. Since energy is a fundamental quantity, if it could be measured, it could help provide a fuller description of the source activity. Even though complete energy information is not directly available, it is nonetheless worthwhile to study bioelectrical activity within the context of electromagnetic energy propagation.

From electrodynamics, electromagnetic energy propagation is described by the Poynting vector S which is defined in terms of the electric and magnetic fields, E and B, as

$$S = \frac{1}{4 π} E \times B . \qquad ( 1 )$$

The Poynting vector has units of energy/(time x area) and it quantifies the flow of electromagnetic energy. Electromagnetic energy flowing into a volume either does work within the volume or increases the energy density stored within the volume. Poynting's theorem is a statement of this energy conservation principle. In differential form it is expressed as

$$\frac{\partial u}{\partial t} + ∇ \cdot S = W \qquad ( 2 )$$

where w is the work done by the fields and u is the electromagnetic energy density stored in the fields. It can be shown from Maxwell's equations that the work done is given by

W=−J·E (3)

where J is the current density and that

$$\frac{\partial u}{\partial t} = \frac{1}{4 π} ( ɛ \frac{\partial E}{\partial t} \cdot E + \frac{1}{μ} \frac{\partial B}{\partial t} \cdot B ) \qquad ( 4 )$$

where ε is the dielectric constant and μ is the magnetic permeability within the volume.

Equation (4) defines the power density and it measures the change in stored energy density due to electromagnetic energy delivery. As with voltage, the time dependence of the power density at a particular measurement location provides information about the underlying source activity. The challenge is to determine the time behavior of the power density from measured quantities. An important observation is that when a physical system responds to a pulse of energy, all energy terms are excited. Even if a system could be initialized so that all of the activity was contained in single term in the power density equation, i.e., Equation (4) above, the activity would quickly be distributed throughout all terms. This is certainly true on the timescale of biomedical processes. Thus, evaluating even a single term in Equation (4) provides valuable information on pulsed activity. With this is mind, the task is to determine the time dependence of an accessible term in the power density equation, i.e., Equation (4) above.

The electric field can be expressed as

$$E ( r , t ) = - ∇ φ - \frac{1}{c} \frac{\partial A}{\partial t} \qquad ( 5 )$$

where φ(r, t) is the electric potential and A(r, t) is the magnetic vector potential.

It follows that a term in power density will be of the form

$$( \frac{\partial }{\partial t} ∇ φ ) \cdot ∇ φ . \qquad ( 6 )$$

If∇φ is a pulse propagating in time, then φ is also a pulse with the same overall propagation behavior but with a different envelope function. At a particular measurement location r, the temporal onset of the pulse is the same regardless of which functional form is observed. Thus the quantity

$$SVP ( t ) ≡ \frac{\partial φ}{\partial t} φ \qquad ( 7 )$$

An alternative signal that also captures the temporal behavior of Eq. (6) follows from the observation that φ(r, t) is actually of the form φ(r−vt) for non-dispersive signals. A time derivative of such a pulse gives the same functional behavior as a gradient. Even for dispersive pulses, the time derivative and spatial gradient functional forms provide similar temporal information. Thus the time behavior of Eq. 6 is also captured by

$$SVP 2 ( t ) ≡ \frac{{\partial }^{2} φ}{\partial {t}^{2}} \frac{\partial φ}{\partial t} \qquad ( 8 )$$

can be used to probe the pulsed temporal behavior of an important term in the power density equation, i.e., Equation (4) above. What is advantageous about this term is that it can be easily calculated through the measured voltage signal.

It is constructive to also apply the analysis used in Equations (5)-(7) above to the work term in Equation (4). Use is made of Ohm's law which states

J=σE. (9)

where σ is the conductivity within the volume. It then follows that the quantity

φ² (10)

exhibits the time dependence of a term in the work done by the fields. Since φ and φ² contain essentially the same information, within the energy framework, studying a voltage measured in time is conceptually equivalent to studying the work done by the electromagnetic fields. But as Poynting's theorem states, this does not explore the full result of electromagnetic energy delivery. It is advantageous to study information gained from the other physical mechanism—energy storage in the fields. The quantity in Equation (7) does not capture the entire process but it does make a significant step towards providing a window on energy storage. An attractive property of Equation (7) is that it provides additional information about source activity without further measurements and minimal extra computation. It is also observed that Equation (7) is the time derivative of Equation (10).

Circuit Example

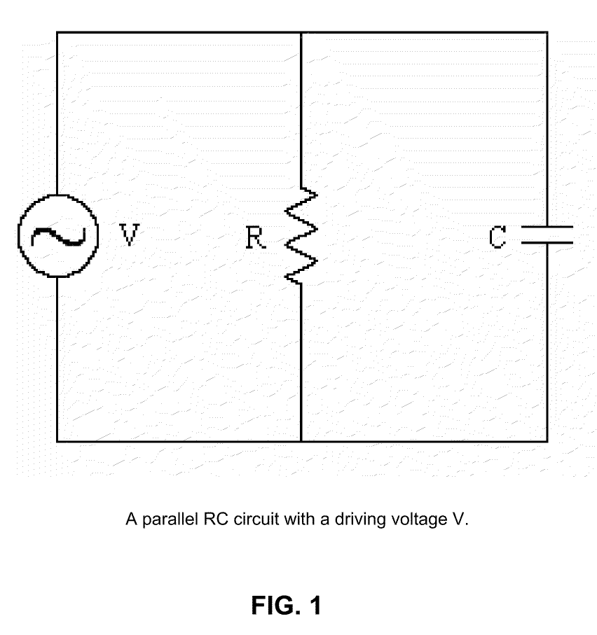

A simple circuit example illustrates the power concepts. FIG. 1 shows an energy source driving a simple parallel RC circuit. From basic circuit theory, the instantaneous power I′ being delivered by a voltage source, V(t), is given by

$$P ( t ) = \frac{{V}^{2}}{R} + C \frac{d V}{d t} \cdot V . \qquad ( 11 )$$

Within a circuit framework, the power delivered by the voltage source goes into a resistive term and a capacitive term. It is instructive, though, to view Equation (11) as a specific case of the general conservation principle defined in Equation (2). Within the energy framework, the power delivered goes into work and storage terms. Here, the work done is simply Joule heating of the resistor and is quantified by the resistive term. The change in stored energy density of Equation (2) is solely capacitive energy storage. This system also has the general property that a pulse of source activity will be reflected immediately in all power terms.

In this system, the power density of Equation (4) is completely characterized by the term of interest in Equation (7). Similarly, the work term is completely characterized by Equation (10). Actual physical systems are more complicated than a simple circuit. The work and storage components typically involve a greater range of physical processes but the terms in Equations (7) and (10) are always present. The quantity in Equation (10) is commonly studied but the quantity in Equation (7) is also physically meaningful.

Signal Processing

Even though Equation (7) was derived from physical principles, it can be viewed within the context of signal processing. As previously stated, biomedical activity often exhibits pulsed behavior. If the voltage signal exhibits pulsed behavior, then it is obvious from its functional form that the quantity in Equation (7) will do so also. In fact, this quantity will enhance a pulse takeoff in the voltage signal. From a signal processing point of view, this term can be thought of as filtering a signal φ with its own derivative. Although this is not a standard signal processing technique, it is apparent that this will enhance transitions in the original signal.

Another observation is that Equation (7) provides an inherent high pass filter on the original signal φ. This is especially helpful in studying biomedical systems where background activity is often present in addition to the activity of interest. Consider a measured voltage φ.

φₘ(t)=φᵢ(ωᵢt)+φ_{b}(ω_{b}t) (12)

where φᵢ is the voltage from the source activity of interest and φ_{b} is the voltage from background activity. ωᵢ and ω_{b} are the primary frequency components of the source and background activities respectively. The power going to work behaves as

P_{w}˜φᵢ²+φ_{b}²+ (13)

whereas the power going to storage behaves as

Pₛ˜ωᵢφᵢ²+ω_{b}φ_{b}²+ (14)

It is often the case that the frequency of the activity of interest is greater than the background activity frequency, i.e.,

ωᵢ>ω_{b}· (15)

This is especially true where the activity of interest is a pulse. From Equations (13) and (14) it can be seen that under this condition, the signal of interest is enhanced relative to the background term in the stored power compared to the work power.

In accordance with the present invention, a recorded voltage signal is used to derive the Stored Voltage Power (SVP) of the recorded voltage signal, and then the Stored Voltage Power (SVP) is used to determine the pulse characteristics (e.g., latency, morphology, etc.) of the recorded voltage signal, whereby to analyze underlying biochemical processes. In many cases, this “power method” can provide results which are significantly more reliable than results which are obtained by looking at only voltage signals per se.

### Biomedical Signals: F-waves

The aforementioned power method may be applied to various biomedical signals in order to more accurately determine the attributes of the biomedical signal, including determining the onset of a pulse (i.e., the point in time of a departure from a baseline voltage signal) in order to better assess event onset.

By way of example but not limitation, the aforementioned power method may be applied in the field of nerve conduction studies (NCS) to determine the latency of F-waves.

More particularly, F-waves are late motor waveforms that occur in response to peripheral nerve stimulations. Specifically, F-waves are orthodromic responses to antidromic impulses which potentially re-excite the motor axon. F-waves are recorded distal to the stimulation site and are typically on the order of tens of microvolts in amplitude. The F-wave latency is the time which elapses between the peripheral nerve stimulation and the arrival of the evoked signal at the recording site. Delayed latencies are indicative of pathology along the path of the response. F-wave responses are physiologically variable. Identical stimulations produce F-wave responses which vary in latency, morphology and amplitude. Therefore, in F-wave analysis, it is not possible to signal average for reduction of noise or other background activity.

The F-wave signals may be obtained using the NC-stat® nerve conduction system produced by NeuroMetrix, Inc. of Waltham, Mass. The NC-stat® system consists of a device for stimulating peripheral nerves and acquiring and processing the resultant signals. Preconfigured electrode grids designed for specific anatomical locations are used as the interface to the patient. The F-wave signals are preferably obtained in the course of standard nerve conduction studies under supramaximal conditions, i.e., at a stimulation level sufficient to produce a maximum response amplitude. The signals are preferably acquired with a 4 kHz sampling rate. Measurements may be performed on a range of nerve types.

The NC-stat® processing software contains an algorithm for calculating F-wave latencies on individual voltage signals. The algorithm determines the maximum signal activity and examines prior candidate takeoff points. It chooses the best takeoff point based on slope and amplitude criteria compared to prior activity. The algorithm does not assign a latency if no possible takeoff point meets sufficient criteria for a well-defined latency. The same algorithm is used for all nerve types but nerve-dependent parameters are used for quantities such as search windows and slope, amplitude and noise thresholds. The algorithm does not need to be modified in any way, or specially “tuned”, to process Stored Voltage Powers (SVPs).

### Results of Processing F-wave Signals using the NC-stat System

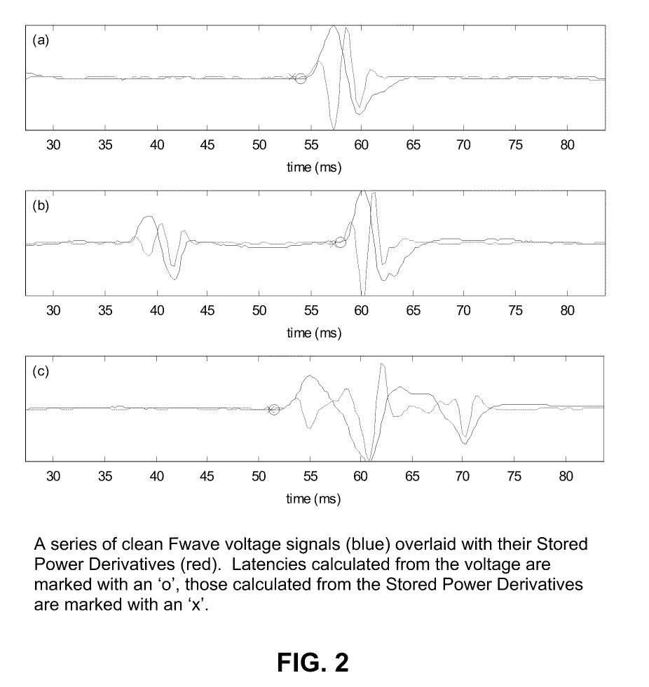

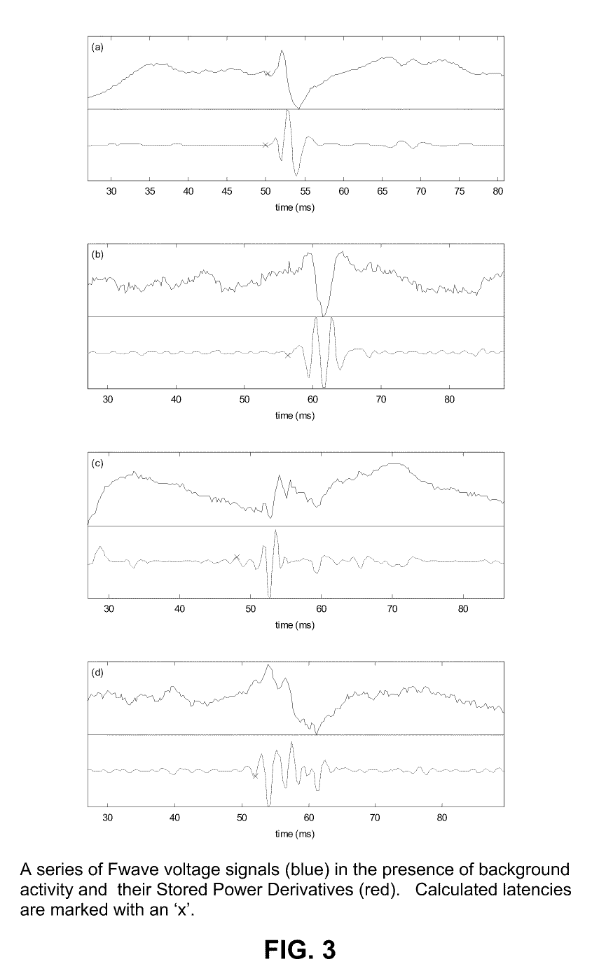

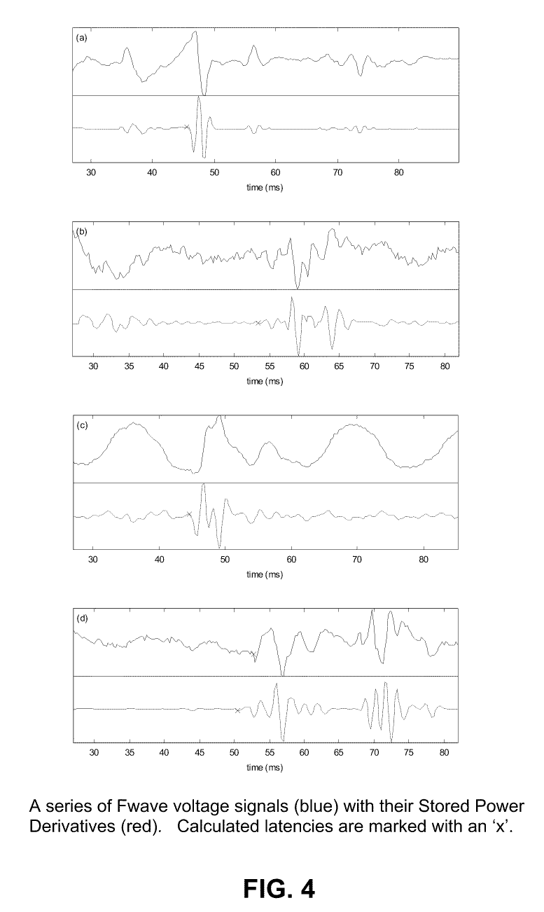

FIG. 2 contains a series of individual F-wave voltage signals overlaid with their respective Stored Voltage Powers (SVPs). The two quantities are scaled so that the absolute maximum of each signal are equal (this scaling holds true for FIGS. 3 and 4 as well). The voltage signals in FIG. 2 are “well-behaved”, in the sense that the baselines are relatively free of noise and other background activity with respect to the F-wave activity levels. The F-wave latencies are easily identified in the acquired voltage signals. As expected from physical and mathematical considerations, the latencies for Stored Voltage Powers (SVPs) are coincident with those of the original voltage signals. In the case where the voltage signal indicates two separate pulses of activity (physiologically possible for these measurements), the corresponding SVP shows consistent activity.

FIG. 2 also illustrates a basic relation between a voltage signal and its SVP, i.e., the frequency of oscillation within the envelope of activity for the SVP is twice that of the voltage. This follows from the functional form of Equation (12). The SVP of a pure sinusoidal signal is also sinusoidal, with exactly twice the original voltage frequency.

If all signals behaved as those in FIG. 2, the SVP would not provide much additional information in F-wave latency determination. The utility of the SVP becomes apparent when the signals depart from a pulse arriving on top of a clean baseline.

FIG. 3 contains a series of individual F-waves and their corresponding SVP's where the background activity is significant compared to the F-wave amplitude in the voltage signal. Graphs (a) & (b) in FIG. 3 are tibial nerve measurements, and graphs (c) and (d) in FIG. 3 are from the peroneal nerve.

The voltage signal in graph (a) in FIG. 3 is representative of an F-wave arriving in the presence of sizeable background activity. The F-wave latency is identifiable but the baseline activity blurs the F-wave pulse. The SVP of the same signal retains the pulse behavior of the F-wave. The latency and duration of the F-wave pulse are more defined than in the voltage signal. The baseline of the SVP signal remains centered and flat. The NC-stat® F-wave latency algorithm provides consistent latencies for both the voltage and the SVP. This signal exemplifies the discussion that the SVP enhances the identification of pulsed activity. These properties of the SVP are found to be consistent across F-wave signals.

Graphs (b)-(d) of FIG. 3 provide examples where the F-wave latency algorithm of the NC-stat® system is not able to ascertain latencies in the voltage signals due to the level of background activity. In each case the SVP contains a well-defined F-wave pulse. The F-wave algorithm of the NC-stat® system is successful in determining latencies when operating on the SVP's. A human eye is able to verify that the SVP latencies are consistent with behavior in the voltage signals.

FIG. 4 contains cases where the SVP's provide information that is difficult to discern in the voltage signals, either by algorithm or human observation. Graph (a) in FIG. 4 shows a voltage measurement where the takeoff of the F-wave in the voltage signal is in the same direction as significant background activity. In this case it is very difficult to ascertain a true latency value with the voltage signal alone. The SVP, however, contains a definite F-wave takeoff Graph (b) of FIG. 4 illustrates the other extreme—the background activity contains numerous transitions making it difficult to identify which is the onset of the F-wave. The SVP exhibits a well-defined takeoff that the algorithm of the NC-stat® system is able to identify. Graph (c) of FIG. 4 shows an F-wave arriving on a background oscillation that precludes an accurate latency determination. The SVP again contains a distinct pulse indicating the true latency. Graph (d) of FIG. 4 is another example of the SVP providing information that is not apparent in the voltage signal alone. The voltage signal shows a distinct onset of what appears to be an F-wave of extended activity. The SVP clearly shows that the activity is comprised of two distinct pulses separated by a time segment which is less than the duration of the pulses. This is not easily discernable in the voltage signal because it is still responding to the first pulse when the subsequent activity arrives. It is often observed that as the voltage signal shows residual response to activity, the SVP exhibits clearer pulse behavior both in onset and in completion.

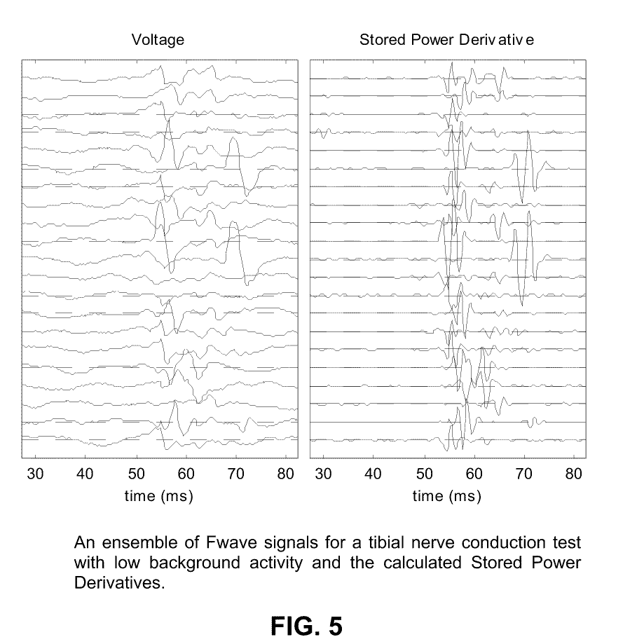

FIGS. 2-4 display individual F-waves from different nerve tests to compare in detail the behavior of a SVP to its voltage signal. Typically, the ensemble of F-waves collected from the series of stimulations of a nerve test is displayed. FIGS. 5-8 display F-wave ensembles with different behavior along with the corresponding Stored Voltage Power (SVP) ensembles. Each figure is scaled so that the absolute maximum of an SVP signal is equal to that of its corresponding voltage.

FIG. 5 displays such an ensemble where the voltage signals contain a low level of background activity. The SVP signals in FIG. 5 exhibit cleaner baselines and more defined F-wave takeoff points.

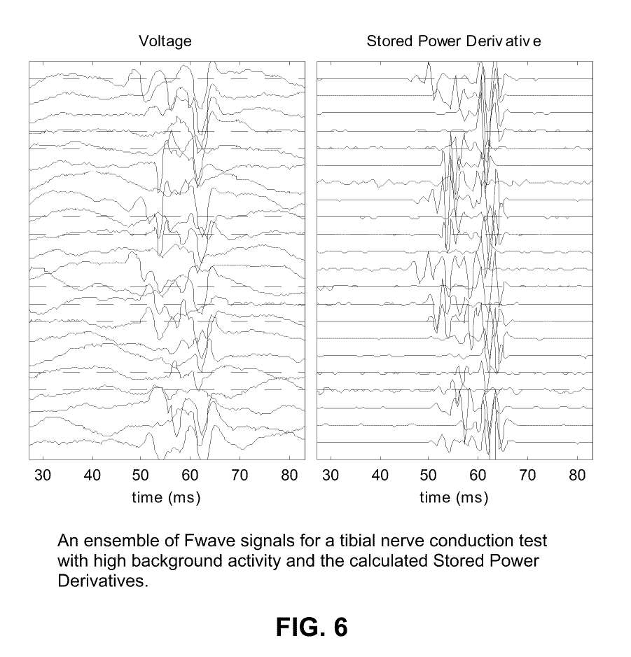

The voltage signals in FIG. 6 contain a high background activity. Even at the higher background levels, however, the SVP signals exhibit flat baselines and well-defined pulse takeoffs.

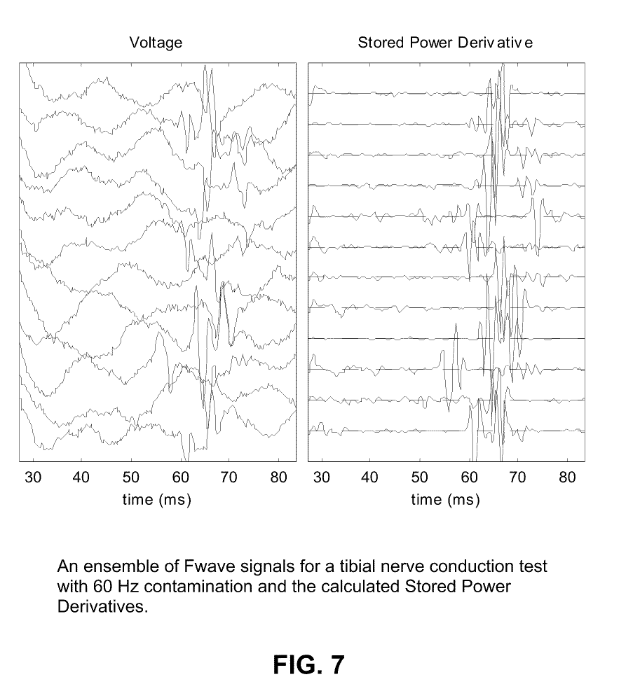

FIG. 7 contains a series of voltage signals in which 60 Hz contamination is present. In the SVP, the F-wave pulses are enhanced and the 60 Hz contamination is minimized. It is important to emphasize that the SVP is the calculation of a physical quantity rather than a processing technique. Analyzing the SVP provides an alternative to filtering the 60 Hz component in the voltage signal. Applying a 60 Hz filter to the voltage removes signal energy and has the potential for changing the true latencies. The calculation of the SVP intrinsically reduces 60 Hz contamination without the disadvantages associated with applying a 60 Hz filter.

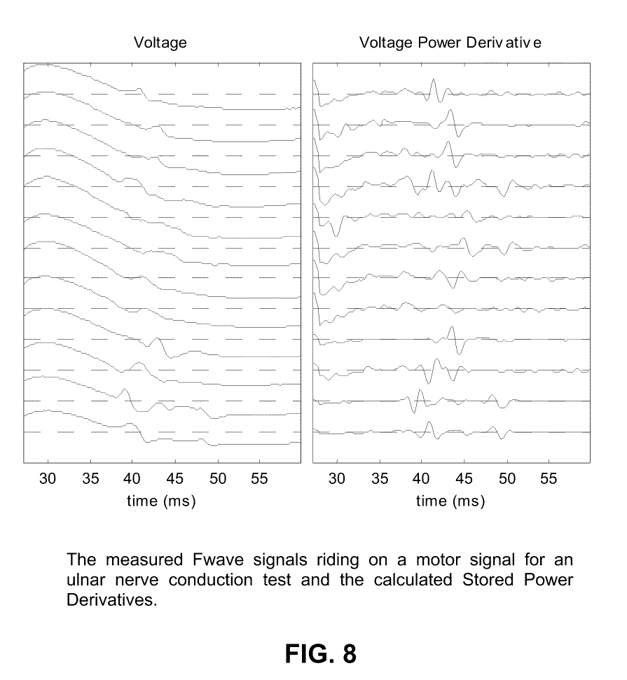

FIG. 8 contains an example of another type of signal contamination that the SVP is able to minimize. In this test, the F-waves arrive in the presence of residual motor activity (because the extent of the voltage is determined by the background and not the F-wave, the SVPs for this figure have been scaled by an additional factor of 2). Again, the SVP enhances the F-wave pulse and helps separate it from the background activity.

Taken together, these examples illustrate the general properties of the Stored Voltage Power as it is applied to F-wave onset determination. The SVP latencies are consistent with the latencies observed in the measured voltage signals where this is observable. The SVP improves the signal to background, whether the background is noise or residual physiological activity. In high activity background signals, the SVP provides information that is not seen in the measured voltage signal.

### Use of the Power Method to Analyze a Wide Range of Biomedical Signals, Including use with Automated Testing Systems

In the foregoing section, it was shown that the use of the Stored Voltage Power (SVP) provides a powerful new tool for determing F-wave onset.

It has also been found that the use of the Stored Voltage Power (SVP) can be applied to determine waveform morphology.

Furthermore, it has also been found that the use of the Stored Voltage Power (SVP) can be applied across a wide range of other biomedical signal activity in order to yield significantly improved results.

Thus it will be seen that the Stored Voltage Power (SVP) provides a powerful new tool for studying biomedical signal activity. It follows from an evaluation of electromagnetic energy propagation and is easily calculated from a measured voltage signal. The Stored Voltage Power (SVP) provides information additional to the measured voltage in investigating pulsed biomedical activity. The Stored Voltage Power (SVP) is particularly useful in discerning energy pulses in the presence of background activity. As was demonstrated above with respect to F-wave processing, the Stored Voltage Power (SVP) allows for the calculation of latencies that are sometimes not possible with the voltage signal alone. And the Stored Voltage Power is equally useful in the analysis of other biomedical signal activity.

Significantly, because use of the Stored Voltage Power (SVP) can provide significantly more distinct pulse onsets, it can enable the use of automated testing devices (e.g., the NC-stat® system for nerve conduction studies) in circumstances where human intervention might otherwise be required. By way of example but not limitation, as discussed above, the power method of the present invention can be used without any modification or any special “tuning” of the automated algorithms used by the NC-stat® system of Neurometrix, Inc. Thus, the use of Stored Voltage Powers (SVPs) is completely compatible with, and can significantly enhance, the NC-stat® system of Neurometrix, Inc. in connection with nerve conduction studies.

### Modifications

It should be understood that many additional changes in the details, materials, steps and arrangements of parts, which have been herein described and illustrated in order to explain the nature of the present invention, may be made by those skilled in the art while still remaining within the principles and scope of the invention.

What is claimed is:

1. A method for studying biomedical processes reflected in a recorded biomedical voltage signal, wherein the method comprises:
obtaining the recorded biomedical voltage signal;
deriving a power signal from the recorded biomedical voltage signal; and
identifying attributes in the corresponding power signal so as to provide information about the underlying biomedical processes.

2. A method according to claim 1 wherein the power signal is obtained by approximating the electric potential term in the Poynting vector for the recorded voltage signal.

3. A method according to claim 2 wherein the power signal is defined to be SVP(t)=φ*dφ/dt where φ is the recorded voltage signal.

4. A method according to claim 3 wherein the power signal is a function of SVP(t).

5. A method according to claim 2 wherein the power signal is defined to be SVP2(t)=d²φ/dt²*dφ/dt where 0 is the recorded voltage signal.

6. A method according to claim 5 wherein the power signal is a function of SVP2(t).

7. A method according to claim 1 wherein the recorded voltage signal is a neurological signal.

8. A method according to claim 7 wherein the neurological signal is acquired in a nerve conduction study.

9. A method according to claim 8 wherein the neurological signal is acquired by electrically stimulating a nerve and recording an evoked response.

10. A method according to claim 1 wherein the attribute comprises pulse onset.

11. A method according to claim 1 wherein the attribute comprises pulse morphology.

12. A system for studying biomedical processes reflected in a recorded biomedical voltage signal, wherein the system comprises:
first apparatus for obtaining and storing a recorded biomedical voltage signal;
second apparatus for deriving a power signal from the recorded biomedical voltage signal; and
third apparatus for identifying attributes in the corresponding power signal so as to provide information about the underlying biomedical processes.

13. A system according to claim 12 wherein the power signal is obtained by approximating the electric potential term in the Poynting vector for the recorded voltage signal.

14. A system according to claim 13 wherein the power signal is defined to be SVP(t)=φ*dφ/dt where φ is the recorded voltage signal.

15. A system according to claim 14 wherein the power signal is a function of SVP(t).

16. A system according to claim 13 wherein the power signal is defined to be SVP2(t)=d²φ/dt²*dφ/dt where φ is the recorded voltage signal.

17. A system according to claim 16 wherein the power signal is a function of SVP2(t).

18. A system according to claim 12 wherein the attribute comprises pulse onset.

19. A system according to claim 12 wherein the attribute comprises pulse morphology.

20. A system according to claim 12 wherein the recorded voltage signal is a neurological signal.

21. A system according to claim 20 wherein the neurological signal is acquired in a nerve conduction study.

22. A system according to claim 20 wherein the neurological signal is acquired by electrically stimulating a nerve and recording an evoked response.

23. A system according to claim 12 wherein the first apparatus comprises a stimulating electrode for applying an electrical stimulus to a patient, and a detection electrode for measuring an evoked response in the patient.
